(19) United States
(12) Patent Application Publication
(10) Pub. No.: US 2026/0306011 A1
(43) Pub. Date: Oct. 8, 2026

(54) DEMENTIA ASSISTING DIAGNOSIS SYSTEM, DEMENTIA ASSISTING DIAGNOSIS METHOD AND COMPUTER PROGRAM PRODUCT THEREOF

(71) Applicant: DONG-MING HSIEH, New Taipei City (TW)

(72) Inventor: DONG-MING HSIEH, New Taipei City (TW)

(21) Appl. No.: 19/173,170

(22) Filed: Apr. 8, 2025

(51) Int. Cl. A61B 5/00; G10L 15/18; G10L 15/26

(52) U.S. Cl. CPC A61B 5/4088; A61B 5/4803; A61B 5/7282; G10L 15/1815; G10L 15/26

(57) ABSTRACT

A dementia assisting diagnosis system, a dementia assisting diagnosis method and a computer program product thereof are disclosed. The dementia assisting diagnosis system reads an audio file obtained by a subject when tested based on a test pattern. The system includes a speech conversion module, an analysis module, an automatic tagging module, and an information output module. The speech conversion module receives the audio file to convert into a text file. The analysis module analyzes a quantity and sequence of a set of keywords in the text file, wherein the set of keywords is obtained through an analysis based on the test pattern. The automatic tagging module generates a grayscale bitmap based on the quantity and sequence of the set of keywords and obtains a continuous heat map using the test pattern. The information output module outputs the continuous heat map and the grayscale bitmap.

### BACKGROUND OF THE INVENTION

### 1. Field of the Invention

The present invention relates to a dementia assisting diagnosis system, a method for dementia assisting diagnosis, and a computer program product, particularly to a dementia assisting diagnosis system, method, and computer program product that can automatically convert an audio file into a chart for interpretation.

### 2. Description of the Related Art

According to statistics from the Alzheimer's Disease International (ADI), the number of dementia patients and the proportion of undiagnosed cases worldwide are steadily increasing. The growing dementia population creates a significant burden on society and consumes vast amounts of medical resources. Preventing and detecting dementia early will become important issues. In the prior art, the diagnosis of early-stage dementia is performed using the Boston Diagnostic Aphasia Examination (BDAE). However, traditional BDAE requires manual administration, which is time-consuming. Additionally, BDAE produces a large amount of text, and interpreting the text remains a significant burden for physicians.

Therefore, there is a need to invent a new dementia assisting diagnosis system, a method for dementia assisting diagnosis, and a computer program product to address the shortcomings of the prior art.

### SUMMARY OF THE INVENTION

The main objective of the present invention is to provide a dementia assisting diagnosis system that has the capability to automatically convert an audio file into a chart for interpretation.

Another main objective of the present invention is to provide a method for dementia assisting diagnosis used in the system described above.

Another main objective of the present invention is to provide a computer program product.

To achieve the above objectives, the dementia assisting diagnosis system of the present invention is used to interpret an audio file obtained from a subject undergoing a test based on a test pattern, wherein the test pattern includes a group of key elements. The dementia assisting diagnosis system includes a speech conversion module, an analysis module, an automatic tagging module, and an information output module. The speech conversion module is configured to receive the audio file and convert it into a text file. The analysis module, electrically connected to the speech conversion module, is configured to analyze the number and sequence of occurrences of a set of keywords within the text file, wherein the set of keywords consists of the group of key elements or synonyms thereof. The automatic tagging module, electrically connected to the analysis module, is configured to generate a grayscale bitmap based on the number and sequence of occurrences of the set of keywords and to generate a continuous heat map using the test pattern. The information output module, electrically connected to the automatic tagging module, is configured to output the continuous heat map and the grayscale bitmap.

The method for dementia assisting diagnosis of the present invention is used in the dementia assisting diagnosis system to interpret an audio file obtained from a subject undergoing a test based on a test pattern, wherein the test pattern includes a group of key elements. The method comprises the following steps: receiving the audio file and converting it into a text file; analyzing the number and sequence of occurrences of a set of keywords within the text file, wherein the set of keywords consists of the group of key elements or synonyms thereof; generating a grayscale bitmap based on the number and sequence of occurrences of the set of keywords, and generating a continuous heat map using the test pattern; and outputting the continuous heat map and the grayscale bitmap.

The computer program product of the present invention is configured to be read and executed from a computer-readable medium to perform the above-described method for dementia assisting diagnosis.

### BRIEF DESCRIPTION OF THE DRAWINGS

All of the objects and advantages of the present invention will become apparent from the following description of the accompanying drawings, which disclose several embodiments of the present invention. It is to be understood that the drawings are to be used for purposes of illustration only, and not as a definition of the invention.

FIG. 1 illustrates a schematic diagram of the architecture of the dementia assisting diagnosis system of the present invention.

FIG. 2 illustrates a schematic diagram of the test pattern of the present invention.

FIG. 3 illustrates a schematic diagram of the grayscale bitmap of the present invention

FIG. 4 illustrates a schematic diagram of the continuous heat map of the present invention.

FIG. 4A illustrates another variation of the schematic diagram of the continuous heat map of the present invention.

FIG. 5 illustrates a flowchart of the steps of the method for dementia assisting diagnosis of the present invention.

### DETAILED DESCRIPTION OF THE PREFERRED EMBODIMENTS

Hereafter, the technical content of the present invention will be better understood with reference to preferred embodiments.

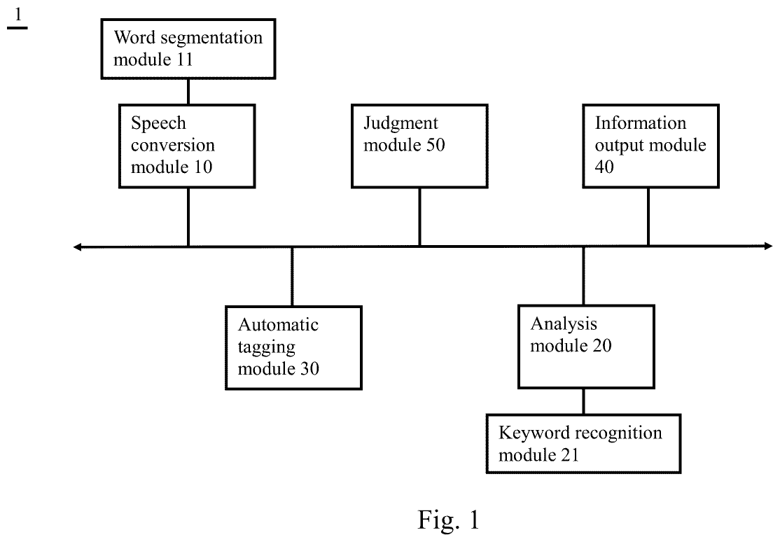

Please refer to FIG. 1, which illustrates a schematic diagram of the architecture of the dementia assisting diagnosis system of the present invention.

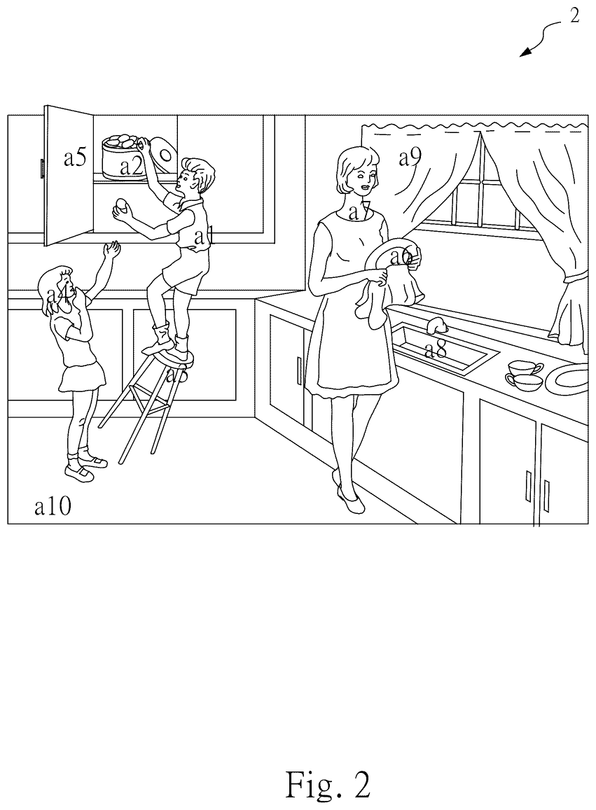

In one embodiment of the present invention, the dementia assisting diagnosis system 1 is a system for evaluating based on the Boston Diagnostic Aphasia Examination (BDAE), used to interpret an audio file obtained from a subject undergoing a descriptive speech test based on a test pattern 2. As shown in FIG. 2, FIG. 2 illustrates a schematic diagram of the test pattern of the present invention. The subject views an image related to “a child stealing cookies while the mother does not notice” (cookie theft image) and is asked to describe the elements, actions of people, and the location depicted in the image. However, the test pattern 2 of the present invention is not limited to the cookie theft image in FIG. 2. Thus, the dementia assisting diagnosis system 1 can interpret the audio file recorded during the subject's test. It should be noted that the dementia assisting diagnosis system 1 can be a computer program product stored on a computer-readable medium to be read and executed, such as in the form of an app installed on a smartphone. However, the invention is not limited to the aforementioned forms.

In one embodiment of the present invention, the dementia assisting diagnosis system 1 includes a speech conversion module 10, an analysis module 20, an automatic tagging module 30, an information output module 40, and a judgment module 50, with each module electrically connected to one another. The speech conversion module 10 is configured to receive the audio file and convert it into a text file. In this embodiment of the present invention, the speech conversion module 10 also includes a word segmentation module 11, which is used to analyze the word boundaries in the audio file and parse the text file to obtain a set of words. Since Chinese sentences do not separate words with spaces like in English, the word segmentation module 11 is required to break down the vocabulary and identify the meaning of each word in the paragraph, thereby identifying multiple words.

The analysis module 20 is electrically connected to the speech conversion module 10, and is used to analyze the number and sequence of occurrences of a set of keywords within the text file. The set of keywords is derived based on the test pattern 2. In one embodiment of the present invention, the analysis module 20 also includes a keyword recognition module 21, which is used to determine, using a related word list, a group of key elements, and a word vector model, whether the set of words obtained from the text file matches the set of keywords by applying an association algorithm. For example, when a word is a key element a1~a10 or a synonym of the key element a1~a10, it is defined as a matching keyword. The group of key elements is related to the elements of each subject in the test pattern 2. Using the cookie theft image in FIG. 2 as an example, the key elements a1~a10 can be summarized based on the narrative keywords of people, places, and things into ten key elements a1~a10: “boy, cookie, chair, girl, cabinet, washing dishes, mother, sink, outside the window, kitchen.” These ten key elements a1~a10 can be derived by a physician from past test records related to the test pattern 2, but the present invention is not limited to this method for deriving the key elements a1~a10. The related word list includes words associated with the group of key elements a1~a10, and is established based on synonyms listed in a dictionary. The data source for the dictionary can be obtained from the Ministry of Education's website, but the invention is not limited to this source. The words in the related word list can also be expanded using an association algorithm. In one embodiment of the present invention, the word vector model is the Word2vec model, but the invention is not limited to this. The Word2vec model is a two-layer neural network that, after learning from large amounts of text data, reconstructs the words based on their context and converts them into mathematical vectors. The simplified word vectors can be used to calculate the distance between similar word vectors, thus obtaining semantically similar words. Since the technology of the word vector model is not the focus of improvement in this invention, it will not be further elaborated here.

In one embodiment of the present invention, the association algorithm is defined as follows:

$${W}_{1} = \{ {P}_{1} , {P}_{2} , … , {P}_{N - 1} \}$$

$${W}_{2} = A \times \{ {W}_{1} \} + b$$

$${W}_{3} = \{ v \vert v ∈ {w}_{1} and v ∈ {w}_{2} \} + b$$

Where W1 represents the narrative content keywords from the test pattern 2 test record provided by the physician, W2 represents the key elements a1~a10 summarized in one embodiment of the present invention, where A is a matrix and b is a bias vector, used to determine the transformation rule. W3 represents the synonyms of the key elements a1~a10 from the dictionary, which includes a set of elements that belong to both the W1 vector and the W2 vector. Thus, the maximum range of the distance metric for synonyms can be defined as:

$$d = max \{ dist ( \vec{V} ( {w}_{2} ) , \vec{V} ( {w}_{3} ) ) \}$$

Where the word vector [symbol] is consistent with the Word2vec model, and dist( [symbol] (w₂), [symbol] (w₃)) represents the distance metric between [symbol] (w₂) and [symbol] (w₃).

Next, assume that the vector x of the input word is an n-dimensional vector, and W2 is a set of n-dimensional vectors. Therefore, the distance metric of the input word can be defined as:

$$D ( x ) = dist ( \vec{V} ( x ) , \vec{V} ( {w}_{2} ) )$$

If the distance metric of the above-mentioned word is greater than or equal to the maximum range of the synonym distance metric d, then the vector x is not included within the vector dimensions between W2 and W3, meaning that this word does not belong to the related words of the key elements a1~a10. If the distance metric of the above-mentioned word is less than the maximum range of the synonym distance metric d, then the vector x is included within the vector dimensions between W2 and W3, meaning that this word belongs to the related words of the key elements a1~a10. In one embodiment of the present invention, the association algorithm can calculate whether any of the words obtained from the text file match the set of keywords, i.e., whether they match the key elements a1~a10 or the synonyms of the key elements a1~a10.

In one embodiment of the present invention, the automatic tagging module 30 is electrically connected to the analysis module 20 and is used to generate a grayscale bitmap 3 based on the number and sequence of occurrences of the set of keywords in the text file, and to generate a continuous heat map 4 using the test pattern 2. Finally, the information output module 40 is electrically connected to the automatic tagging module 30 and is used to output the continuous heat map 4 and the grayscale bitmap 3 generated by the automatic tagging module 30 for interpretation by the physician.

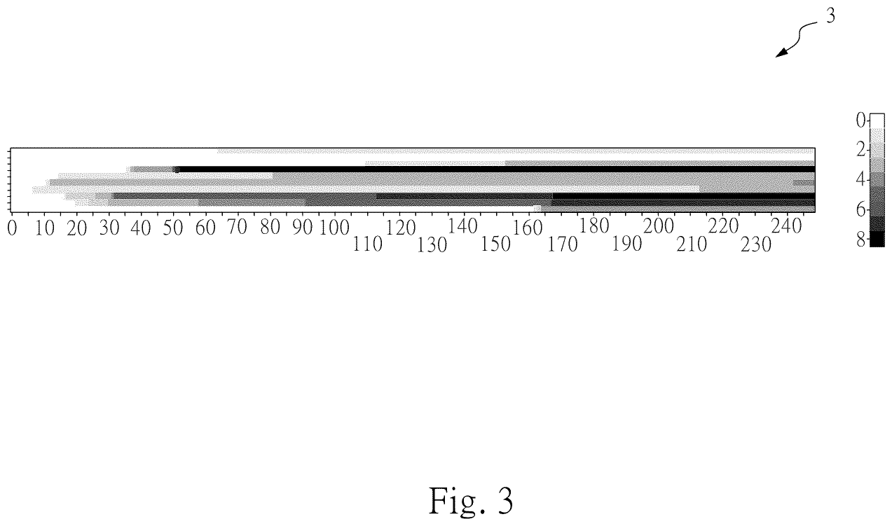

Please refer to FIG. 3, which illustrates a schematic diagram of the grayscale bitmap of the present invention.

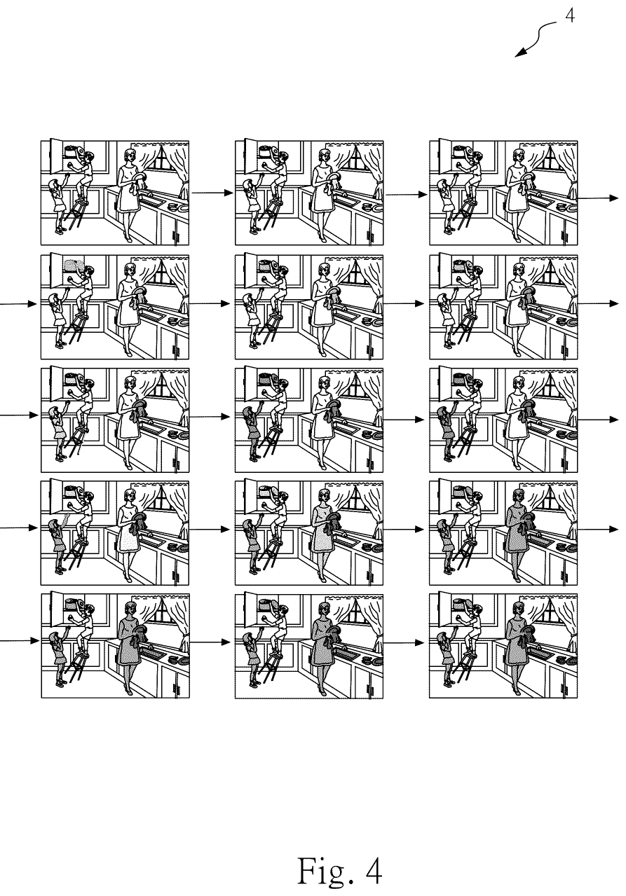

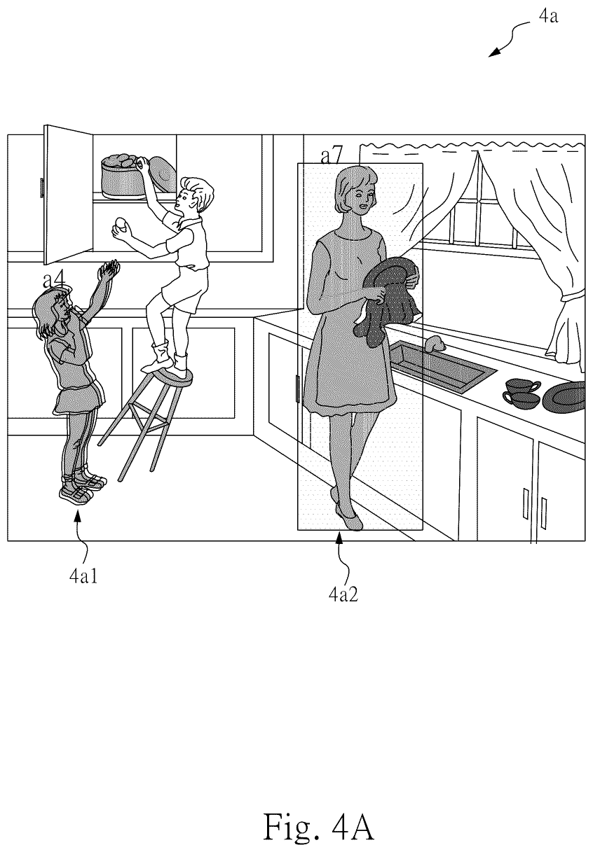

The automatic tagging module 30 orders all the keywords mentioned by the subject during the testing process along the x-axis based on their word sequence, and then compares the similarity of each key element a1~a10 using cosine similarity. After the comparison, the results are arranged in a one-dimensional array along the y-axis, generating the grayscale bitmap 3. This allows for a clearer record of the occurrence of each key element a1~a10. To avoid having too many color gradients that are difficult for the human eye to differentiate and to prevent saturation of the color scale from occurring too quickly due to low frequency, in one embodiment of the present invention, 8 occurrences are used as the baseline for the grayscale bitmap 3. If any key element a1~a10 is mentioned more than 8 times, it is recorded as 8 occurrences. The continuous heat map 4 is also generated based on this standard. It should be noted that, although FIGS. 3 and 4 use varying shades of gray to represent different frequencies, other colors can be used to achieve a better distinction. On the other hand, the automatic tagging module 30 can also generate two types of grayscale bitmaps 3: one for the physician, which includes clear coordinate axes and a grayscale scale, and another that adjusts the word length to a fixed setting, which serves as input data for training the judgment module 50 of the subsequent dementia assisting diagnosis system 1. This allows the judgment module 50 to analyze the grayscale bitmap 3.

Additionally, please refer to FIG. 4, which illustrates a schematic diagram of the continuous heat map of the present invention.

The automatic tagging module 30 marks each time a key element a1~a10 is mentioned by the subject at each time point on the test pattern 2. For example, each time the subject mentions the key element 6 “washing dishes”, it is marked in the corresponding area of the test pattern 2. Other mentioned key elements are marked sequentially. Thus, the final result can be visualized on a timeline, producing the 15 continuous heat maps 4 as shown in FIG. 4, based on the test pattern 2. The continuous heat map 4 can be output as a GIF animation file, but the invention is not limited to this. In the prior art, only the final marked results of the test pattern 2 could be seen, but these final results do not necessarily represent whether the subject is normal or abnormal. By using the continuous heat map 4, the order and frequency of the key elements mentioned by the subject can be clearly understood.

For example, if each key element is marked an average number of times in the final results of the test pattern 2, the subject is more likely to be classified as normal. However, if the continuous heat map 4 shows that the subject repeatedly mentions certain key elements in a short period of time, the subject may be classified as abnormal. On the other hand, if certain key elements are marked more frequently in the final results of the test pattern 2, the subject is more likely to be classified as abnormal. However, if the continuous heat map 4 shows that the subject mentions all the key elements in a balanced manner, the subject may still be classified as normal. Therefore, the information output module 40 outputs the grayscale bitmap 3 and the continuous heat map 4 for the physician to interpret.

In another embodiment of the present invention, the keyword recognition module 21 can use Natural Language Processing (NLP) algorithms to analyze the word vectors. After performing frequency statistics on the related words, the module can carry out “conversion of auxiliary words” and “association interpretation of synonyms and antonyms” to obtain the vector relationships between meanings, thereby determining whether the subject has mentioned auxiliary words or antonyms related to the set of keywords. The automatic tagging module 30 then interprets and classifies the results, drawing different effects on the continuous heat map 4 to represent the auxiliary words or antonyms of the set of keywords.

Please refer to FIG. 4A, which illustrates a schematic diagram of another variation of the continuous heat map of the present invention.

The continuous heat map 4a shown in FIG. 4A can be one of the 15 continuous heat maps 4 mentioned earlier. For example, when the subject is supposed to mention the key element a4 “girl” but only says auxiliary words like “um, ah, oh, this is, what,” it indicates that the subject is unable to provide substitute vocabulary for the characters and events in the test pattern 2 (i.e., the cookie theft image). In this case, the keyword recognition module 21 identifies these as auxiliary words related to the key element a4 “girl.” The automatic tagging module 30 can display the graphic of “girl” using the oscillatory effect 4a1. Additionally, if the subject is supposed to mention the key element a7 “mother” but only says “father,” an antonym, the keyword recognition module 21 identifies this as an antonym related to the key element a7 “mother.” The automatic tagging module 30 can display the graphic of “mother” using the blurring effect 4a2. If the subject mentions a synonym of the key element “mother,” the automatic tagging module 30 will maintain the graphic of “mother” clearly displayed. It should be noted that the oscillatory effect 4a1 and the blurring effect 4a2 are only illustrative examples; the invention does not limit the use of these effects to represent the interpretation of auxiliary words or antonyms on the continuous heat map 4a.

On the other hand, the dementia assisting diagnosis system 1 can also utilize the judgment module 50 for interpretation. The judgment module 50 can be a MobileNetV2 classifier, which is a convolutional neural network (CNN) architecture. Convolutional neural networks are deep learning models commonly used for solving image recognition and classification problems. Additionally, the judgment module 50 can further analyze the grayscale bitmap 3 based on an age and an education level of the subject. Therefore, after training, the judgment module 50 can automatically determine whether the subject is normal or abnormal, serving as a reference for the physician's interpretation.

It should be noted that the modules of the dementia assisting diagnosis system 1 can be implemented as hardware devices, software programs combined with hardware devices, or firmware combined with hardware devices, for example, one or more computer program products can be stored on one or more computer-readable media to be read and executed to achieve the functions of the present invention. However, the invention is not limited to the aforementioned methods. Furthermore, this embodiment only illustrates the preferred embodiments of the present invention. To avoid redundancy, not all possible variations and combinations are elaborated upon in detail. However, one skilled in the art should understand that not all of the modules or components described are necessarily required. To implement the present invention, other more detailed conventional modules or components may also be included. Each module or component may be omitted or modified as needed, and there may be other modules or components between any two modules.

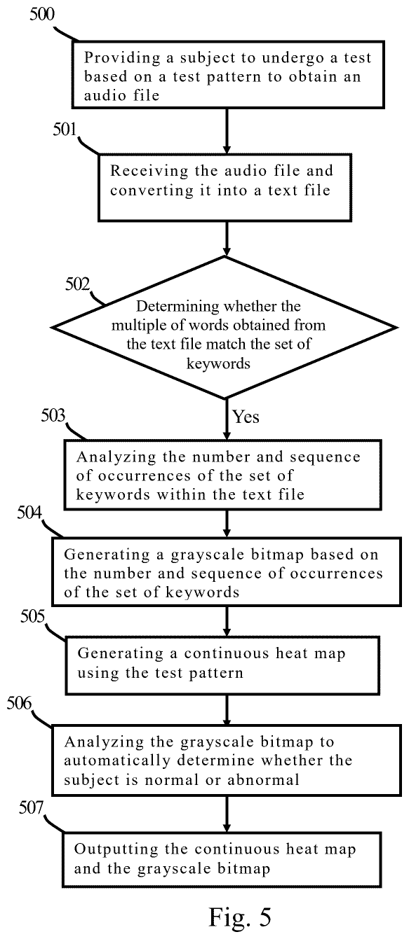

Please refer to FIG. 5, which illustrates a flowchart of the steps of the dementia assisting diagnosis method of the present invention. It should be noted that although the dementia assisting diagnosis system 1 is used as an example to explain the dementia assisting diagnosis method of the present invention, the method is not limited to being used with the same structure of the dementia assisting diagnosis system 1 described above.

First, step 500 is performed: providing a subject to undergo a test based on a test pattern to obtain an audio file.

First, the subject is provided with a test pattern 2 to view and is asked to describe the elements in the image, what the people are doing, and the location depicted in the image. This allows for the generation of an audio file. The test pattern 2 includes a group of key elements a1~a10.

Next, step 501 is performed: receiving the audio file and converting it into a text file.

The speech conversion module 10 is used to receive the audio file and convert it into a text file, while simultaneously utilizing the word segmentation module 11 to analyze the word boundaries in the audio file, in order to distinguish the multiple of words in the text file.

Then, step 502 is performed: determining whether the multiple of words obtained from the text file match the set of keywords.

The keyword recognition module 21 is used to use a related word list, the group of key elements a1~a10, and a word vector model to apply an association algorithm to calculate the maximum range of the distance metric for synonyms. This is used to determine whether the distance metric of each word in the multiple of words obtained from the text file is greater than or equal to the maximum range of the distance metric for synonyms. This method is used to determine whether the multiple of words in the text file match the set of keywords, i.e., whether they correspond to the key elements a1~a10 or synonyms of the key elements a1~a10.

If none of the set of keywords match, the dementia assisting diagnosis system 1 will naturally be unable to produce valid results, which may indicate that the subject is abnormal or that an unexpected error occurred. However, if there are words in the text file that match the set of keywords, step 503 is performed: analyzing the number and sequence of occurrences of the set of keywords within the text file.

At this point, the analysis module 20 analyzes the number and sequence of occurrences of the set of keywords in the text file, while simultaneously expanding the related word list. The keyword recognition module 21 can also interpret auxiliary words or antonyms related to the set of keywords.

In this way, the automatic tagging module 30 can perform step 504: generating a grayscale bitmap based on the number and sequence of occurrences of the set of keywords.

The automatic tagging module 30 uses the word sequence of all the keywords mentioned by the subject during the testing process as the x-axis. It then performs similarity comparison using cosine similarity for each key element a1~a10, and arranges the results in a one-dimensional array to be used as the y-axis, thereby generating the grayscale bitmap 3.

And then perform step 505: generating a continuous heat map using the test pattern.

At the same time, the automatic tagging module 30 marks each time a keyword is mentioned on the test pattern 2. If, in step 503, the keyword recognition module 21 confirms that the multiple of words are auxiliary words or antonyms related to the keywords, the automatic tagging module 30 can apply special oscillatory or blurring effects on the pattern based on the auxiliary words or antonyms related to the keywords. In the end, a continuous heat map 4 of the test pattern 2 can be generated according to the timeline.

Next, step 506 is performed: analyzing the grayscale bitmap to automatically determine whether the subject is normal or abnormal.

After training, the judgment module 50 can automatically determine whether the subject is normal or abnormal. The judgment module 50 can also further consider an age and an education level of the subject to analyze the grayscale bitmap 3.

Finally, step 507 is performed: outputting the continuous heat map and the grayscale bitmap.

Finally, the information output module 40 is used to output the continuous heat map 4 and the grayscale bitmap 3 for the physician's interpretation.

It should be noted that the steps of the dementia assisting diagnosis method of the present invention are not limited to the above order. As long as the objectives of the present invention can be achieved, the order of the steps can be modified.

As can be seen from the above description, the dementia assisting diagnosis system 1 can directly convert the subject's audio file into a grayscale bitmap 3 and a continuous heat map 4 for interpretation by the physician, or the judgment module 50 of the dementia assisting diagnosis system 1 can first analyze the subject's condition. This effectively addresses the shortcomings of the prior art.

It is noted that the above-mentioned embodiments are only for illustration. It is intended that the present invention cover modifications and variations of this invention provided they fall within the scope of the following claims and their equivalents. Therefore, it will be apparent to those skilled in the art that various modifications and variations can be made to the structure of the present invention without departing from the scope or spirit of the invention.

What is claimed is:

1. A dementia assisting diagnosis system, configured to interpret an audio file generated from a subject undergoing a test based on a test pattern, wherein the test pattern includes a group of key elements, the dementia assisting diagnosis system comprising:
a speech conversion module configured to receive the audio file and convert it into a text file;
an analysis module, electrically connected to the speech conversion module, configured to analyze the number and sequence of occurrences of a set of keywords within the text file, wherein the set of keywords comprises the group of key elements or synonyms thereof;
an automatic tagging module, electrically connected to the analysis module, configured to generate a grayscale bitmap based on the number and sequence of occurrences of the group of keywords and to generate a continuous heat map using the test pattern; and
an information output module, electrically connected to the automatic tagging module, configured to output the continuous heat map and the grayscale bitmap.

2. The dementia assisting diagnosis system as claimed in claim 1, wherein the speech conversion module includes:
a word segmentation module configured to analyze word boundaries in the audio file, thereby parsing the text file to obtain a multiple of words.

3. The dementia assisting diagnosis system as claimed in claim 2, further comprising:
a keyword recognition module configured to apply an association algorithm with a related word list, the group of key elements, and a word vector model to determine whether the multiple of words obtained from the text file match the set of keywords.

4. The dementia assisting diagnosis system as claimed in claim 3, wherein the keyword recognition module determines whether a distance metric of each word in the multiple of words is greater than or equal to a maximum range of the distance metric of synonyms, thereby confirming whether the multiple of words are synonyms of the set of keywords.

5. The dementia assisting diagnosis system as claimed in claim 3, wherein the keyword recognition module further confirms whether the multiple of words are antonyms of the set of keywords, and the automatic tagging module, based on this confirmation, determines whether to apply a blurring effect to the continuous heat map.

6. The dementia assisting diagnosis system as claimed in claim 3, wherein the keyword recognition module further confirms whether the multiple of words are auxiliary words, and wherein the automatic tagging module, based on this confirmation, determines whether to apply an oscillatory effect to the continuous heat map.

7. The dementia assisting diagnosis system as claimed in claim 3, wherein the related word list is established based on related synonyms found in a dictionary.

8. The dementia assisting diagnosis system as claimed in claim 1, further comprising:
a judgment module configured to analyze the grayscale bitmap, thereby automatically determining whether the subject is normal or abnormal.

9. The dementia assisting diagnosis system as claimed in claim 8, wherein the judgment module further analyzes the grayscale bitmap based on an age and an education level of the subject.

10. A method for dementia assisting diagnosis, used in a dementia assisting diagnosis system, for interpreting an audio file obtained from a subject undergoing a test based on a test pattern, wherein the test pattern includes a group of key elements, the method comprising the following steps:
receiving the audio file and converting it into a text file;
analyzing the number and sequence of occurrences of a set of keywords within the text file, wherein the set of keywords comprises the group of key elements or synonyms thereof;
generating a grayscale bitmap based on the number and sequence of occurrences of the set of keywords, and generating a continuous heat map using the test pattern; and
outputting the continuous heat map and the grayscale bitmap.

11. The method for dementia assisting diagnosis as claimed in claim 10, further comprising the step of analyzing the word boundaries in the audio file to parse the text file and obtain a multiple of words.

12. The method for dementia assisting diagnosis as claimed in claim 11, further comprising the following step:
using a related word list, the group of key elements, and a word vector model to apply an association algorithm to determine whether the multiple of words obtained from the text file match the set of keywords.

13. The method for dementia assisting diagnosis as claimed in claim 12, further comprising the following step:
determining whether the distance metric of each word in the multiple of words is greater than or equal to the maximum range of the distance metric of synonyms, to confirm whether the multiple of words are synonyms of the set of keywords.

14. The method for dementia assisting diagnosis as claimed in claim 12, further comprising the following steps:
confirming whether the multiple of words are antonyms of the set of keywords; and
If the multiple of words are antonyms of the set of keywords, applying a blurring effect to the continuous heat map.

15. The method for dementia assisting diagnosis as claimed in claim 12, further comprising the following steps:
confirming whether the multiple of words are auxiliary words; and
If the multiple of words are auxiliary words, applying an oscillatory effect to the continuous heat map.

16. The method for dementia assisting diagnosis as claimed in claim 12, further comprising the following step:
establishing the related word list based on related synonyms found in a dictionary.

17. The method for dementia assisting diagnosis as claimed in claim 10, further comprising the following step:
analyzing the grayscale bitmap to automatically determine whether the subject is normal or abnormal.

18. The method for dementia assisting diagnosis as claimed in claim 17, further comprising the following step:
analyzing the grayscale bitmap based on an age and an education level of the subject.

19. A computer program product, configured to be read and executed from a computer-readable medium to perform the method as claimed in claim 10.
